(19) United States
(12) Patent Application Publication
(10) Pub. No.: US 2009/0149946 A1
(43) Pub. Date: Jun. 11, 2009

(54) STENT HAVING AT LEAST ONE BARB AND METHODS OF MANUFACTURE

(75) Inventor: Christopher G. Dixon, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/325,420

(22) Filed: Dec. 1, 2008

Related U.S. Application Data
(60) Provisional application No. 60/992,484, filed on Dec. 5, 2007.

(51) Int. Cl. A61F 2/06

(52) U.S. Cl. 623/1.36; 623/1.15; 623/1.32

(57) ABSTRACT

The present embodiments provide a barbed stent having at least one integrally-formed barb. In one embodiment, the barbed stent comprises a stent having at least one segment forming a strut. A slit is formed in the strut, preferably such that the slit is disposed partially but not entirely through the strut. A barbed portion is formed extending from the strut, whereby the slit separates the barbed portion from a remainder of the strut. The barbed portion then may be bent at an angle with respect to the strut, and a sharpened tip suitable for engaging tissue may be formed, for example, by grinding an end region of the barbed portion.

### PRIORITY CLAIM

This invention claims the benefit of priority of U.S. Provisional Application Ser. No. 60/992,484, entitled “Stent Having at Least One Barb and Methods of Manufacture,” filed Dec. 5, 2007, the disclosure of which is hereby incorporated by reference in its entirety.

### BACKGROUND

The present embodiments relate generally to apparatus and methods for treating vascular conditions, and more specifically, to a barbed stent.

Stents may be inserted into an anatomical vessel or duct to maintain or restore patency in a formerly blocked or constricted passageway. For example, stents may be used to maintain patency in a vessel following a balloon angioplasty procedure. Other stents may be used for different procedures, for example, stent-grafts may employ at least one stent configured to hold a graft in an open configuration to treat an aneurysm. Additionally, stents may be coupled to one or both ends of a graft, and then extend proximally or distally away from the graft, e.g, to engage a healthy portion of a vessel wall away from a diseased portion of an aneurysm to provide endovascular graft fixation.

Stents may be either self-expanding or balloon-expandable. Self-expanding stents may be delivered to a target site in a compressed configuration and subsequently expanded by removing a compression sheath. In such embodiments, a shape-memory alloy such as nitinol may be employed to cause the stent to return to a predetermined configuration upon removal of the sheath.

If balloon-expandable stents are used, the stent may be delivered and deployed using a catheter having proximal and distal ends and a balloon disposed on the distal end of the catheter. The stent may be coupled to the balloon during insertion towards a target location. The delivery system comprises a smaller delivery profile than the diameter of the vessel into which the stent is implanted. The catheter may be inserted over a wire guide into a vessel or duct and advanced until the stent is aligned at the target site. The stent then may be deployed by inflating the balloon to expand the stent diameter, whereby the stent engages the target site.

Stents may be manufactured using materials such as plastic or metal, and may comprise a variety of configurations. For example, stents may comprise a wire-mesh, coil or helical shape, or a slotted tube configuration. One commonly-employed stent design is known as a “Z-stent” or Gianturco stent. The Gianturco stent typically comprises a series of substantially straight segments interconnected by a series of bent segments. The bent segments may comprise acute bends or apices. The stent is arranged in a zigzag configuration in which the straight segments are set at angles relative to each other and are connected by the bent segments.

In order to ensure their attachment at the target site, many known stents employ barbs configured to engage tissue. The barbs may protrude in a radially outward direction relative to a longitudinal axis of the stent. The barbs may comprise a sharpened end configured to engage or penetrate a portion of the tissue to provide a fixation point to reduce movement of the stent relative to the vessel.

Stent barbs may comprise various shapes and sizes. The barbs may be formed integrally with the stent or attached thereto. Barbs may be formed intregrally with a stent as an extension of a strut, in which no other joint or connection exists. Integral barbs also may be formed by laser cutting the stent into a desired pattern in order to produce the desired barb configuration, for example, by forming the barbs from a single ribbon, sheet or tube stock.

By contrast, barbs that are attached to a stent may comprise small protrusions or “coil-stacks” that are affixed to an exterior surface of the stent, for example, using soldering or welding techniques. In the latter embodiment, a first end of the barb is affixed to the stent and a second end of the barb may comprise a sharpened end configured to engage or penetrate tissue.

In view of the above, it would be desirable to provide a stent having at least one structurally stable, reliable, and easy to manufacture barb.

### SUMMARY

The present embodiments provide a barbed stent having at least one barb. In one embodiment, the barbed stent comprises a stent having at least one segment forming a strut. A slit is formed in the strut, preferably such that the slit is disposed partially but not entirely through the strut. The slit may be formed at one or more angles with respect to a longitudinal axis of the strut. A barbed portion is formed extending from the strut, whereby the slit separates the barbed portion from a remainder of the strut. The barbed portion then may be bent at an angle with respect to the strut to form an integral barb.

The barbed stent may comprise multiple integrally-formed barbs. The barbs may be formed at acute, orthogonal or obtuse angles with respect to a longitudinal axis of the stent. Moreover, multiple barbs may be oriented in the same proximal or distal direction to facilitate engagement with a target site, or alternatively, different barbs may face in different longitudinal directions.

Preferred method steps also are provided for manufacturing a stent in accordance with the present embodiments. Optionally, a reinforcing member, such as solder, may be disposed within a portion of the slit and adhered to a portion of the barbed portion to enhance the structural stability of the barbed portion. Further, a sharpened tip suitable for engaging tissue may be formed, for example, by grinding an end region of the barbed portion.

Advantageously, the integral barb may be easier to manufacture and encompass enhanced structural stability relative to other barbs that are integrally formed into a stent. Moreover, the integral barb may comprise manufacturing and structural advantages over barbs that are not integrally formed, but rather attached to the stent, for example, by welding or soldering.

A barbed stent in accordance with the present embodiments may be used alone or in conjunction with a stent-graft. If used in conjunction with a stent-graft, the barbed stent may be disposed substantially internal to the graft material such that the sharpened tip protrudes through the graft material to engage tissue. Alternatively, the barbed stent may be disposed external to the graft material such that it does not perforate the graft material. A fixation stent coupled to a stent-graft also may comprise one or more barbs formed in accordance with the techniques described herein.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIGS. 1-2 are side views of a stent having at least one integrally formed barb in accordance with a first embodiment before and after manufacture, respectively.

FIGS. 3-4 are side views illustrating a method of manufacturing a stent having at least one integrally formed barb.

FIG. 5 is a side view of an optional manufacturing step to form a reinforced integrally formed barb.

FIG. 6 is a side view of an optional manufacturing step to form a sharpened integrally formed barb.

FIG. 7 is a side view of a stent-graft having at least one fixation stent comprising at least one integrally-formed barb.

FIG. 8 is a side view illustrating an alternative method of manufacturing a stent having at least one integrally formed barb.

FIG. 9 is a side view illustrating a further alternative method of manufacturing a stent having at least one integrally formed barb.

### DETAILED DESCRIPTION OF THE PREFERRED EMBODIMENTS

In the present application, the term “proximal” refers to a direction that is generally toward a physician during a medical procedure, while the term “distal” refers to a direction that is generally toward a target site within a patient's anatomy during a medical procedure.

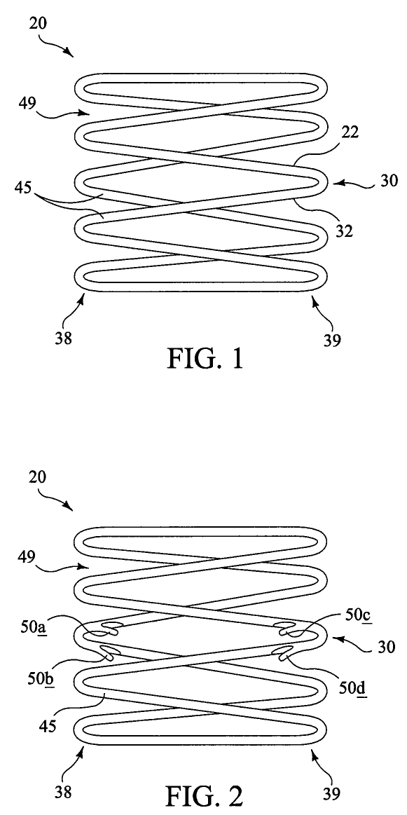

Referring now to FIGS. 1-2, a first embodiment of a stent having at least one integrally formed barb is described. As depicted in FIG. 1, the stent 20 may comprise a generally zig-zag shape having a proximal end 38 and a distal end 39. The stent 20 may be formed from a single wire comprising a plurality of substantially straight first segments 22 and second segments 32 having a plurality of bent segments 30 disposed therebetween. Each bent segment 30 generally comprises an apex, as shown in FIG. 1. The plurality of substantially straight first segments 22 and second segments 32 form a series of struts 45. As will be explained in further detail below, one or more integral barbs 50 may be formed in one or more struts 45.

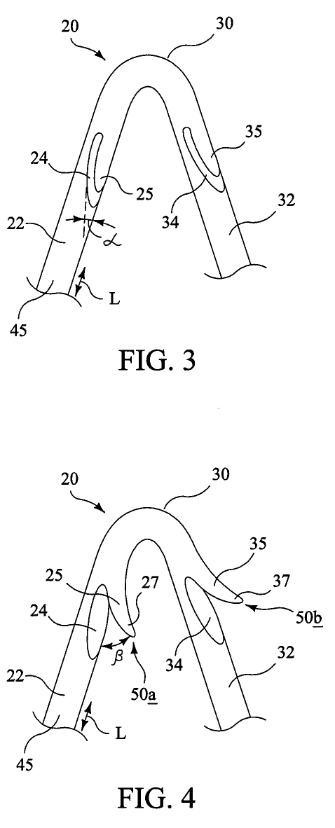

Referring to FIG. 2, at least one integral barb of the stent 20 is formed, preferably gusing one or more of the techniques described with respect to FIGS. 3-6 below. In the embodiment of FIG. 2, integral barbs 50a and 50b at the proximal end 38 of the stent have been bent in a distal direction to form distally-oriented integral barbs, while integral barbs 50c and 50d at the distal end 39 of the stent have been bent in proximal directions to form proximally-oriented integral barbs. As will be apparent, while four integral barbs 50a-50d have been depicted in FIG. 2, the stent 20 may comprise any number of integral barbs formed using the techniques described herein. Further, while the integral barbs 50a-50d are shown being disposed near the bent segments 30 of the stent 20, the integral barbs alternatively may be disposed closer to central portions of the struts 45 and/or may be disposed closer to the apices of the bent segments 30.

Moreover, the integral barbs may be formed to collectively point in the same direction, or alternatively, at least one integral barb may be oriented in a proximally-oriented direction while at least one other integral barb is oriented in a distally-oriented direction, as depicted in FIG. 2. As will be explained further below, the angular orientation of the integral barbs with respect to the strut 45 may be varied, for example, to be tailored for particular anatomical regions.

The stent 20 has a reduced diameter delivery state in which it may be advanced to a target location within a vessel, duct or other anatomical site. The stent 20 further has an expanded deployed state in which it may be configured to apply a radially outward force upon the vessel, duct or other target location, e.g., to maintain patency within a passageway. In the expanded state, fluid flow is allowed through a central lumen 39 of the stent 20.

Referring now to FIG. 3, a portion of the stent 20 is shown prior to formation of an integral barb. In an exemplary first manufacturing step, at least one notch or slit 24 is formed in the first segment 22, e.g., by laser cutting. As shown in FIG. 3, the provision of the slit 24 forms a barbed portion 25. The slit 24 effectively separates the barbed portion 25 from a remainder of the first segment 22. Similarly, a second notch or slit 34 may be formed in the second segment 32 to separate a barbed portion 35 from a remainder of the second segment 32.

The slit 24 may be formed in the strut 45 at an angle α with respect to a longitudinal axis L of the strut 45, as shown in FIG. 3. The angle α may be anywhere between 0 and 90 degrees, and preferably is about 5 to 45 degrees. As the angle α is reduced, the length of the slit 24 may be increased without cutting through the diameter of the strut 45.

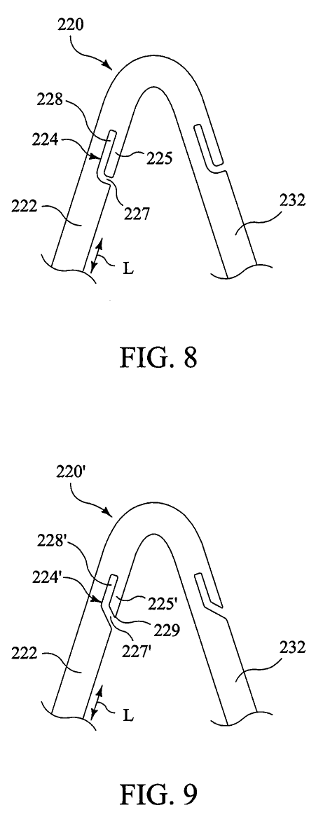

Alternatively, as explained below with respect to FIGS. 8-9, other slit formations may be provided. For example, the slit 24 may comprise a first region that is substantially orthogonal or at another angle with respect to the longitudinal axis L of the strut, and then a second portion that is substantially parallel to the longitudinal axis L of the strut.

As shown in FIG. 4, the end regions of the barbed portions 25 and 35 that are furthest from the struts 45 of the first and second segments 22 and 32 may form barb tips 27 and 37, respectively. The barbed portion 25 may be bent in a direction away from the first segment 22, such that the barb tip 27 is angled away from the first segment 22 to form an angle β with respect to the longitudinal axis L of the strut 45, as shown in FIG. 4. Similarly, the barbed portion 35 may be bent in a direction away from the second segment 32, such that the barb tip 37 is angled away from the second segment 32. The bending of the barbed portions 25 and 35 therefore forms a plurality of integral, protruding barbs 50a and 50b, which are configured to engage tissue.

The angle β, formed between the barb tip 27 and the strut 45, preferably comprises an acute angle, and more preferably in the range of about 5 to 85 degrees. As best seen in FIG. 4, the angle β depicted in the present embodiment is about 50 degrees. Alternatively, the angle β may comprise a substantially orthogonal angle, such that the barb tip 27 points at a substantially perpendicular angle with respect to the strut 45. Further, it is possible that the barbed portion 25 is curved backward greater than 90 degrees, such that barb tip 27 may point at an obtuse angle with respect to the strut 45.

The bending step may be performed manually or using any suitable mechanical device in order to bend the barbed portions 25 and 35 into the desired configurations. The preferred angle may be tailored to the procedure for which the stent 20 may be used. For example, in certain procedures it may be desirable to have an acute angle, while in others it may be desirable to have a substantially orthogonal or obtuse angle.

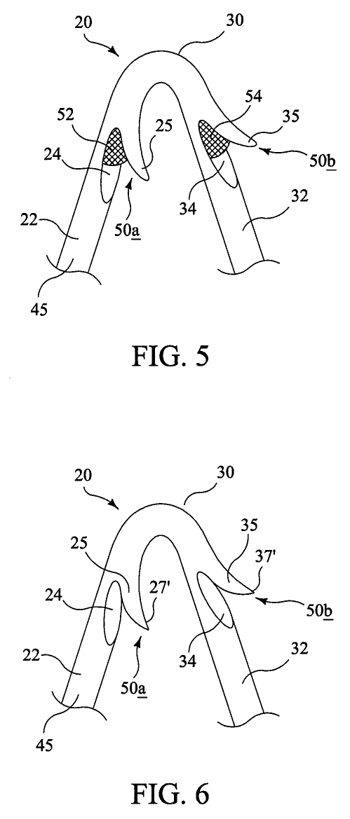

Referring now to FIG. 5, an optional manufacturing step is described to form a reinforced integral barb. At least one reinforcing member 52 is disposed at least partially within the slit 24 and configured to reinforce the attachment between a portion of the barbed portion 25 and the strut 45 of the first segment 22. The reinforcing member 52 preferably is disposed at least partially within the slit 24 and adhered to at least a portion of the barbed portion 25 and the strut 45, as shown in FIG. 5. Similarly, a reinforcing member 54 may be disposed at least partially within the slit 34 and adhered to at least a portion of the barbed portion 35 to reinforce the barbed portion 35.

In a preferred embodiment, the reinforcing members 52 and 54 may comprise solder. During manufacture, after the barbed portions 25 and 35 have been bent into the desired angular configurations as set forth in FIG. 4 above, the solder may be applied within at least a portion of the slits 24 and 34 and/or may be applied to cover a portion or the entire circumference of the barbed portions 25 and 35. Moreover, the solder may be applied around a portion or the entire circumference of the struts of the first and second segments 22 and 32. While it may be desirable to cover each of the aforementioned locations to enhance the structural stability of the integral barbs 50a and 50b, full coverage may not be employed to reduce the profile of the device.

Referring now to FIG. 6, in an optional manufacturing step, the barb tips 27 and 37 may be sharpened to enhance their engagement with target tissue. In one embodiment, the barb tips 27 and 37 may be sharpened by grinding the exterior surfaces of the barbed portions 25 and 35, respectively. The sharpening step forms sharpened edges 27′ and 37′ in the exterior surfaces of the barbed portions 25 and 35, respectively. Therefore, integral barbs 50a and 50b having sharpened tips may be disposed at desired angular configurations to engage tissue.

While additional sharpening of the barbed portions is preferred, it will be apparent that integral barb tips that are not sharpened, such as the barb tips 27 and 37 of FIG. 4, also may be designed or configured to engage tissue. Further, it will be apparent that both manufacturing steps in FIGS. 5-6 may be used, thereby providing integral barbs 50a and 50b having both reinforcing members 52 and 54 and sharpened tips 27′ and 37′, respectively.

Advantageously, the integral barbs 50 of the stent 20 may be easier to manufacture and encompass enhanced structural stability relative to other barbs that are integrally formed into a stent. Additionally, the stent 20 may comprise manufacturing advantages over barbs that are not integrally formed, but rather attached to the stent, for example, by welding or soldering of the barbs to the stent. Such welding or soldering techniques may be difficult to perform and the stability of the barb once again may be compromised. By contrast, in the present embodiments, solder or another adherent is only optionally applied if it is desirable to enhance the attachment of the integral barbed portion, rather than apply a separate barb entirely.

It will be appreciated that while the stent 20 is shown comprising a zig-zag configuration, the stent may alternatively comprise any number of shapes, as long as at least one solid region having a sufficient diameter or surface area, preferably in the form of a strut, is provided along a portion of the stent. If a solid portion is provided, then any of the techniques described above with respect to FIGS. 3-6 may be employed to manufacture one or more integral barbs in accordance with the present embodiments. For example, the stent 20 may comprise a support structure having a pattern of interconnected struts.

The stent 20 may be made from numerous metals and alloys. In a preferred embodiment, the stent 20 comprises a self-expanding nitinol or stainless steel stent. Alternatively, the stent 20 may comprise other materials such as cobalt-chrome alloys, amorphous metals, tantalum, platinum, gold and titanium. The stent 20 also may be made from non-metallic materials, such as thermoplastics and other polymers. The structure of the stent may also be formed in a variety of ways to provide a suitable intraluminal support structure. For example, stents may be made from a woven wire structure, a laser-cut cannula, individual interconnected rings, or any other type of stent structure that is known in the art. Regardless of the particular construction of the stent, it is usually desirable for the stent to be flexible in several directions, including both radial and axial flexibility.

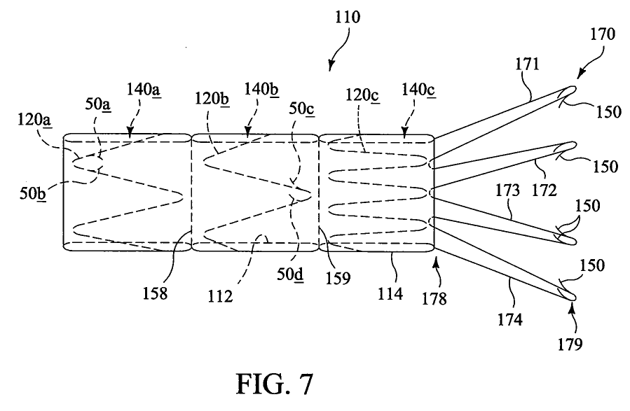

Referring now to FIG. 7, one or more of the stents 20 provided in accordance with the present embodiments also may be used in conjunction with a stent-graft having one or more stents. For example, a stent-graft 110 may be provided substantially in accordance with a stent-graft described in applicant's commonly-owned, pending application Ser. No. 11/716,818, filed Mar. 12, 2007, the disclosure of which is hereby incorporated by reference in its entirety.

The stent-graft 110 may comprise an inner graft 112 and an outer graft 114. A first pocket 140a, second pocket 140b and third pocket 140c may be formed by circumferentially attaching the inner graft 112 to the outer graft 114 at attachment points 158 and 159 to thereby separate adjacent pockets. The stents 120a-120c may be held within the pockets 140a-140c, respectively, as shown in FIG. 7.

The stents 120a-120c preferably are provided in accordance with the stent 20 described above, such that each stent comprises at least one slit formed in a strut, wherein the slit is disposed partially but not entirely through the strut and a barbed portion extends from the strut to form an integral barb. For example, in the embodiment of FIG. 7, the first stent 120a may comprise first and second integral barbs 50a and 50b, which are oriented in radially outward directions at an acute angle, such that they each point distally. In this embodiment, the integral barbs 50a and 50b of the first stent 120a are formed in lateral surfaces of struts of the first stent 120a such that their tips point towards one another. By contrast, the second stent 120b comprises integral barbs 50c and 50d, which are oriented radially outward and point in proximal directions. Unlike the integral barbs 50a and 50b, the tips of the integral barbs 50c and 50d are formed such that they are substantially parallel to one another.

As will be apparent, all of the barbs 50a-50d may point in the same proximal or distal direction, or one or more barbs may be oriented in different directions, as depicted in FIG. 7. Moreover, if the stents 120a-120c are disposed substantially between the inner graft 112 and the outer graft 114, any of the integral barbs 50a-50d may protrude through the outer graft 114 to engage tissue.

Many different types of graft materials may be used for the inner graft 112 and the outer graft 114. Common examples of graft materials currently used include expandable polytetrafluoroethylene (ePTFE), polytetrafluoroethylene (PTFE), Dacron, polyester, fabrics and collagen. However, graft materials may be made from numerous other materials as well, including both synthetic polymers and natural tissues, including small intestine submucosa (SIS).

Alternatively, the barbed stent 20 described herein may be used in conjunction with a single piece of graft material. The barbed stent 20 may be coupled to graft material, for example, using sewing techniques. If the stent 20 is disposed internal to the graft material, then an integral barb 50 may protrude through the graft material to engage tissue. If the stent 20 is disposed external to the graft material, the integral barb 50 will not perforate the graft material but rather will directly engage tissue.

In a still further embodiment, a barbed stent provided in accordance with the present embodiments may be used for endovascular graft fixation. For example, fixation stent 170 comprises a series of zig-zag segments 171-174, each having a proximal end 178 and a distal end 179. The proximal ends 178 of the segments 171-174 may be coupled to a distal end of the stent-graft 110, for example, by sewing. The distal ends 179 of the segments of the fixation stent 170 may be flared in a radially-outward direction, e.g, to engage a healthy portion of a vessel wall. The distal ends 179 that engage the vessel wall may comprise one or more integrally-formed barbs 150, which may be substantially identical to the barbs 50 formed in accordance with the techniques described above.

In one embodiment, the stent-graft 110 having the fixation stent 170 may be used to treat an aneurysm, such as an abdominal aortic aneurysm. In this case, the fixation stent 170 extends beyond a diseased portion of the aneurysm and is flared radially outward to engage a healthy region of tissue. As will be apparent, the stent-graft 110 may comprise alternative configurations suitable for a particular application, for example, the proximal end of the stent-graft 110 may be bifurcated such that one leg is configured to be disposed in a contralateral iliac artery while the other leg is configured to be disposed in an ipsilateral iliac artery.

The stent 20 and the stent-graft 110 of the aforementioned embodiments may be delivered into a vessel, duct, or other anatomical site using a suitable deployment system or introducer. In one embodiment, an introducer as described in PCT application WO98/53761, entitled “A Prosthesis and a Method and Means of Deploying a Prosthesis,” which is incorporated herein by reference in its entirety, may be used to deploy the stent or stent-graft. PCT application WO98/53761 describes a deployment system for an endoluminal prosthesis whereby the prosthesis is radially compressed onto a delivery catheter and is covered by an outer sheath. To deploy the system, the operator slides or retracts the outer sheath over the delivery catheter, thereby exposing the prosthesis. The prosthesis expands outwardly upon removal of the sheath. The operator can directly manipulate the sheath and the delivery catheter, which provides the operator with a relatively high degree of control during the procedure. Further, such delivery devices may be compact and may have a relatively uniform, low-diameter radial profile, allowing for atraumatic access and delivery.

The delivery and deployment device used to deploy the stent 20 and the stent-graft 110 may optionally include deployment control mechanisms. For example, a proximal control mechanism may releasably retain the proximal end of the stent-graft 110 and a distal control mechanism may releasably retain the distal end of the stent-graft 110. The proximal and distal control mechanisms may comprise one or more trigger wires that releasably couple the proximal and distal ends of the stent-graft 110 to the delivery catheter. Various prosthesis retention devices, configurations, and methods of use are disclosed in PCT application WO 98/53761, previously incorporated by reference. While the above-referenced PCT application described one system for delivering and deploying the stent 20 and the stent-graft 110, other suitable delivery and deployment systems may be used to deliver a barbed stent or stent-graft manufactured in accordance with the embodiments and techniques described hereinabove.

Referring now to FIGS. 8-9, side views showing alternative techniques for forming a slit into a strut are shown. In FIG. 8, the stent 220 comprises at least one notch or slit 224 formed in a first segment 222, e.g., by laser cutting. As shown in FIG. 8, the provision of the slit 224 forms a barbed portion 225. The slit 224 effectively separates the barbed portion 225 from a remainder of the first segment 222. In the embodiment of FIG. 8, the slit 224 comprises a first region 227 that is substantially orthogonal with respect to the longitudinal axis L of the strut, and further comprises a second region 228 that is substantially parallel to the longitudinal axis L of the strut. Similarly, a second notch or slit may be formed in the second segment 232 to separate a barbed portion from a remainder of the second segment.

In FIG. 9, an alternative stent 220′ comprises at least one notch or slit 224′ formed in a first segment 222, e.g., by laser cutting. As shown in FIG.9, the provision of the slit 224′ forms a barbed portion 225′. The slit 224′ effectively separates the barbed portion 225′ from a remainder of the first segment 222. In the embodiment of FIG. 9, the slit 224′ comprises a first region 227′ that is formed at an angle with respect to the longitudinal axis L of the strut, and further comprises a second region 228′ that is substantially parallel to the longitudinal axis L of the strut. Using this technique, a sharpened tip 229 may be formed at the end of the barbed portion 225′. Similarly, a second notch or slit may be formed in the second segment 232 to separate a barbed portion from a remainder of the second segment.

The barbed portions 225 and 225′ of the embodiments of FIGS. 8-9, respectively, may be subsequently manufactured using one or more of the techniques described above with respect to FIGS. 4-6. For example, the barbed portions 225 and 225′ may be bent in a direction away from the first and second segments 222 and 232, reinforced using a reinforcing member such as solder, and/or the tips of the barbed portions 225 and 225′ may be further sharpened, e.g., by grinding.

While various embodiments of the invention have been described, it will be apparent to those of ordinary skill in the art that many more embodiments and implementations are possible within the scope of the invention. Accordingly, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. A barbed stent for use in an endoluminal procedure, comprising:
a stent having at least one segment forming a strut;
a slit formed in the strut; and
a barbed portion extending from the strut, wherein the slit separates the barbed portion from a remainder of the strut.

2. The barbed stent of claim 1, wherein the slit is formed in the strut at an angle of about 5 to about 45 degrees with respect to a longitudinal axis of the strut.

3. The barbed stent of claim 1, wherein the slit comprises a first region that is substantially orthogonal with respect to a longitudinal axis of the strut and further comprises a second region that is substantially parallel to the longitudinal axis of the strut.

4. The barbed stent of claim 1, wherein the slit comprises a first region that is formed at an angle with respect to a longitudinal axis of the strut and further comprises a second region that is substantially parallel to the longitudinal axis of the strut.

5. The barbed stent of claim 1, wherein the barbed portion is formed at an acute angle with respect to the strut by bending the barbed portion with respect to the strut.

6. The barbed stent of claim 1, wherein the barbed portion comprises a sharpened tip.

7. The barbed stent of claim 1 further comprising a reinforcing member disposed at least partially within the slit and further disposed between a portion of the barbed portion and the strut.

8. The barbed stent of claim 7, wherein the reinforcing member comprises solder.

9. The barbed stent of claim 1, wherein the stent comprises a Z-shaped stent.

10. The barbed stent of claim 1, wherein the barbed stent comprises at least two barbed portions, wherein a first barbed portion is formed in a proximally-oriented direction and a second barbed portion is formed in a distally-oriented direction.

11. A barbed stent for use in an endoluminal procedure, comprising:
a stent having at least one segment forming a strut;
a slit formed in the strut, wherein the slit is formed in the strut at an angle of about 5 to about 45 degrees with respect to a longitudinal axis of the strut; and
a barbed portion extending from the strut, wherein the slit separates the barbed portion from a remainder of the strut, and wherein an end region of the barbed portion is bent at an angle with respect to the remainder of the strut.

12. The barbed stent of claim 11, wherein the end region of the barbed portion comprises a sharpened tip configured to engage tissue.

13. The barbed stent of claim 11 further comprising a reinforcing member disposed at least partially within the slit and further disposed between a portion of the barbed portion and the strut.

14. The barbed stent of claim 13, wherein the reinforcing member comprises solder.

15. The barbed stent of claim 11, wherein the stent comprises a Z-shaped stent.

16. The barbed stent of claim 11, wherein the stent comprises a fixation stent having proximal and distal regions, wherein the proximal region is coupled to a distal end of a stent-graft, and wherein the distal region comprises at least one barbed portion configured to engage tissue.

17. A method for forming a barbed stent, comprising:
providing a stent having at least one segment forming a strut;
forming a slit in the strut; and
forming a barbed portion that extends from the strut, wherein the slit separates the barbed portion from a remainder of the strut.

18. The method of claim 17 further comprising forming the barbed portion at an angle with respect to the strut by bending the barbed portion with respect to the strut.

19. The method of claim 17 further comprising sharpening a tip of the barbed portion by grinding at least an end portion of the barbed portion.

20. The method of claim 17 further comprising reinforcing the slit to enhance the attachment between a portion of the barbed portion and the strut.
